(19) United States
(12) Patent Application Publication
(10) Pub. No.: US 2024/0408284 A1
(43) Pub. Date: Dec. 12, 2024

(54) BIOCORRODIBLE SOLID BODY AND METHOD FOR COATING A SOLID BODY

(71) Applicant: BIO-GATE AG, Nürnberg (DE)

(72) Inventor: Peter STEINRÜCKE, Nürnberg (DE)

(73) Assignee: BIO-GATE AG, Nürnberg (DE)

(21) Appl. No.: 18/697,969

(22) PCT Filed: Sep. 29, 2022
(86) PCT No.: PCT/EP2022/077157

§ 371 Date: Apr. 2, 2024

(30) Foreign Application Priority Data
Oct. 5, 2021 (DE) 10 2021 125 789.1

(51) Int. Cl. A61L 31/02; A61L 31/06; A61L 31/08; A61L 31/14; A61L 31/16

(52) U.S. Cl. CPC A61L 31/022; A61L 31/06; A61L 31/088; A61L 31/146; A61L 31/148; A61L 31/16; A61L 2300/404; A61L 2430/02

(57) ABSTRACT

A biocorrodible solid body, in particular in the form of an implant, has a base material and a coating. The coating includes silicon and is applied to the base material in such a way that different corrosion rates are established in different areas of the solid body.

### CROSS-REFERENCE TO RELATED APPLICATIONS

This application is a National Stage Application of PCT/EP2022/077157, filed Sep. 29, 2022, which claims benefit of priority to German Patent Application No. 102021125789.1, filed Oct. 5, 2021, and which applications are incorporated herein by reference. To the extent appropriate, a claim of priority is made to each of the above disclosed applications.

### FIELD OF THE INVENTION

The invention relates to a biocorrodible solid body, in particular in the form of a biocorrodible implant. The invention further relates to a method for coating such a solid.

### PRIOR ART

A method for treating a surface of a metallic implant made of a biodegradable material is known, for example, from WO 2012/007181 A1. In this case, a dispersed system containing apatite powder is used to coat an implant. An AC voltage is applied for the apatite to transform into an oxide layer. The method according to WO 2012/007181 A1 is said to be particularly suitable for coating magnesium-based implants.

Biocorrosion generally refers to the change in materials caused by the activities of living beings, which leads to the destruction of said materials or to damage to objects connected to them or consisting of them. Biocorrosion usually leads to an acceleration of material deterioration. Typical biological causes can be bacteria, fungi, yeast but also higher organisms such as plants. Bacterial biofilms play a predominant role in biocorrosion because of their diversity and robustness at very high organism densities.

An antimicrobial layer material is known from EP 1 790 224 B1, which comprises a biocide layer and a transport control layer covering said biocide layer. An active ingredient in the biocide layer is silver, copper, zinc, ions thereof or metal complexes thereof or a mixture or alloy of the elements mentioned. The transport control layer has a thickness and porosity set to deliver the biocidal agent through the transport control layer in an antimicrobial and non-cytotoxic amount. The transport control layer contains silicon, carbon and oxygen in area-defined proportions. The layer material according to EP 1 790 224 B1 can be applied to a polyurethane surface, for example. Sputtering and plasma polymerization are mentioned as possible methods for producing the transport control layer. Overall, the layer material according to EP 1 790 224 B1 is suitable, among other things, for coating bone nails and other bone implants. The layer material is designed in particular to prevent corrosive attacks from body fluids.

A method described in EP 1 941 918 A2 for producing a corrosion-inhibiting coating on an implant, that is to say a stent, made of a biocorrodible magnesium alloy includes treating an implant surface with an aqueous or alcoholic conversion solution which contains certain ions at a concentration in the range of 0.01 mol/l to 2 mol/l. The aim of the method proposed in EP 1 941 918 A2 is to only temporarily inhibit corrosion.

Another method for producing a corrosion-inhibiting coating on an implant made of a biocorrodible magnesium alloy is described in EP 2 189 170 A1. In this case, an anodic plasma-chemical treatment of an implant surface takes place in an aqueous, fluoride-free electrolyte that contains ammonia, phosphoric acid and boric acid. The proposed method is said to be able to produce a coating with a thickness in the range of 1 μm to 10 μm. The coating is said to be suitable for loading with an active pharmaceutical ingredient.

In EP 2 033 668 A2, which also deals with an implant made of a biocorrodible magnesium alloy, a coating is proposed which contains a biocorrodible polyphosphazene.

WO 2010/017959 A2 describes an implant made of a magnesium alloy in which the porosity increases towards the core. Possible coatings in this case include oxide layers and polymers, for example poly-L-lactic acid, which dissolve within a certain residence time.

A coating composition described in EP 2 726 558 B2 comprises a polysiloxane-based binder matrix. This coating composition further comprises hydrophilic oligomer/polymer units.

### SUMMARY OF THE INVENTION

The invention is based on the object of providing refined options for the targeted biocorrosion of coatings and articles at least partially coated with them compared to the prior art.

The configurations and advantages of the invention explained below in connection with the coating method apply analogously also for the coated article, in particular the coated implant, and vice versa.

The biocorrodible solid body comprises a base material and a coating that at least partially covers it and contains silicon. In particular, this can be a coating of the type described in EP 1 790 224 B1. The coating is applied to the base material in such a way that different corrosion rates are established in different areas of the solid body, whereby parts of the solid can remain uncoated.

The coated solid body can be used either in a living being or in another environment in which degradation of the solid body occurs through biocorrosion. In all cases, the biodegradability can be set by means of the coating in such a way that the process of biocorrosion and thus the formation of degradation products does not progress at a uniform rate of degradation over the entire surface of the solid body. With the use of silicon, this can even be achieved if the elemental composition of the coating is uniform. In particular, different sections of the coating can differ from one another in terms of their porosity, with the porosity having a significant impact on the hydrophilic or hydrophobic properties of the coating. Furthermore, the thickness of the coating may be non-uniform. Plastics, for example polyethylene, can also be mentioned as possible components of the coating. In any case, the different areas of the solid body provided with the coating, in which different corrosion rates are established, differ from one another with regard to at least one of the properties porosity, thickness, hydrophilic/hydrophobic properties and composition.

Just like the coating, the base material can also have a porous structure. The porosity of this structure is also not necessarily uniform throughout the solid body. For example, there can be a higher porosity of the base material in volume areas that are designed for comparatively rapid biocorrosion. This desired effect can be supported by a tuned thickness and/or composition of the coating. In particular, the coating can interfere with the biocorrosion of the base material only to a small extent or—for example, by causing the setting of a certain pH—even promote it.

The biocorrodible solid body can in principle be an article of any desired, not necessarily rigid, geometry. For example, if the biocorrodible solid has an elongated, substantially cylindrical shape, its thickness can vary over the length of the solid body. In a comparable manner, a solid body with a planar shape can also have a non-uniform thickness.

Either a coating with uniform biocorrosion properties or a coating with biocorrosion properties that vary from surface section to surface section can be applied to a solid body with a uniform thickness. The properties that differ depending on the surface section in terms of the biodegradability of the coating can be given either by the coating thickness or by other parameters of the coating. In any case, there are different possibilities of correlation between the biodegradability of the coating on the one hand and the biodegradability of the base material on the other hand, wherein the degradability of the base material can be determined in particular by its thickness, but also by other properties, in particular the material composition and/or porosity.

If there is a positive correlation between the biodegradability of the coating and the biodegradability of the base material, this means that a rapid degradability of the coating in a certain area of the solid body is accompanied by an equally rapid degradability of the base material in the corresponding area. On the contrary, it is also possible that the areas of the solid body with the lowest material thickness should be degraded the latest. This can be achieved by providing the thinnest sections of the solid body with a particularly slowly degradable coating. In this case, there is a negative correlation between the biodegradability of the coating and the biodegradability of the base material, which is particularly impacted by the material thickness.

If the biocorrodible solid body is an implant, the base material can be either a metallic or a non-metallic material. Magnesium is particularly suitable as a metallic material. Examples of possible non-metallic materials include polymers, in particular a polylactide, polysaccharide, polyamide, polyester, PLGA or PCL.

The base material can have a uniform or non-uniform material structure. In both cases, the article generally referred to as a solid body can also be a fibrous or tissue-like product. It is also possible to apply the coating to biological material, such as seeds.

If the biocorrodible solid body is designed as an implant, it can be provided to absorb considerable mechanical loads. This applies, for example, to implants in hand surgery and foot surgery. In such cases, the non-uniform degradation rates can generally serve, on the one hand, to release degradation products, for example hydrogen, at rates that are not too high and, on the other hand, to specifically maintain long-term mechanical resilience in defined areas of the implant.

In general, the solid body, for example the implant, can be coated by applying a biocidal substance and a silicon-containing coating to a base material in a non-uniform manner on various surface sections, which means that biodegradability in individual areas of the solid body, in particular the implant, is set differently. With regard to the biocidal substance that may be contained in the coating, reference is again made to the already mentioned EP 1 790 224 B1.

As part of the coating process, for example, the biocidal substance is first applied to the entire surface of the base material in a uniform manner. In a further method step, the silicon-containing coating is applied to various surface sections in a non-uniform manner.

Alternatively, both the biocidal substance and the silicon-containing coating can be applied to different surface sections of the base material in a non-uniform manner. In both cases it is possible to apply the biocidal substance and the further coating in such a way that the biocidal substance is only released as the coating corrodes.

According to a possible processing, the coating is applied to the base material in such a way that the coating is impermeable to oxygen on at least a partial area of the base material, at least at the beginning of use. In the course of use, i.e. in particular when the implant is worn, increasing oxygen permeability and thus accelerated degradation of the entire solid body can occur.

### BRIEF DESCRIPTION OF THE DRAWINGS

Multiple exemplary embodiments of the invention are explained in more detail below with reference to a drawing. In the figures, in some instances in roughly schematic form:

FIG. 1 shows in the manner of a stylized X-ray image structures of a human hand including an implant,

FIGS. 2 and 3 show sections of the bone structures of the hand according to FIG. 1 with the implant in different stages of corrosion,

FIG. 4 shows a possible structure of a coated, biocorrodible implant,

FIG. 5 shows a further exemplary embodiment of a coated, biocorrodible implant in a schematic sectional view.

### DETAILED DESCRIPTION OF THE INVENTION

Unless otherwise stated, the following explanations refer to all exemplary embodiments. Corresponding parts and parameters are marked with the same reference numerals.

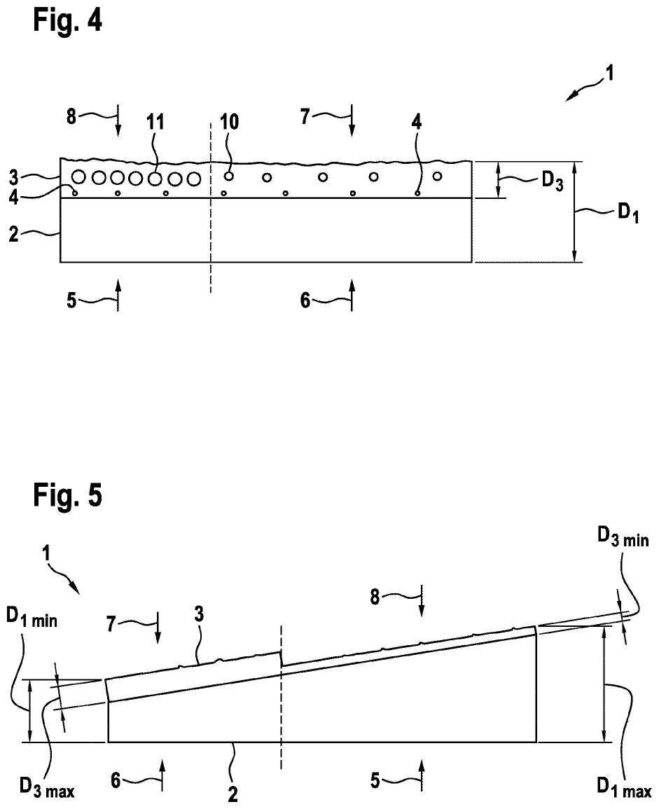

A medical technology implant 1 is designed as a solid body, which is made up of a base material 2, in the present cases a magnesium alloy, and a coating 3 located thereon. The thickness of solid body 1 is generally given as D₁. If solid body 1, i.e. the implant, has a non-uniform thickness, the designation D₁ₘᵢₙ is used for the smallest thickness and D₁ₘₐₓ is used for the largest thickness. The designations D₃, D₃ₘᵢₙ and D₃ₘₐₓ are used for the thickness of coating 3. In FIGS. 4 and 5, the thickness of coating 3 is shown in exaggerated representation. Deviating from the symbolized representations, the entire surface of base material 2 can be coated.

Base material 2 is a biocorrodible material. The actual biocorrosion that occurs when implant 1 is in the patient's body is significantly impacted by the thickness and nature of coating 3. In the present cases, coating 3 contains silicon as the main component and also a biocidal substance 4, which is located directly on the surface of base material 2. In the cases outlined, biocidal substance 4 is applied evenly to the surface of base material 2.

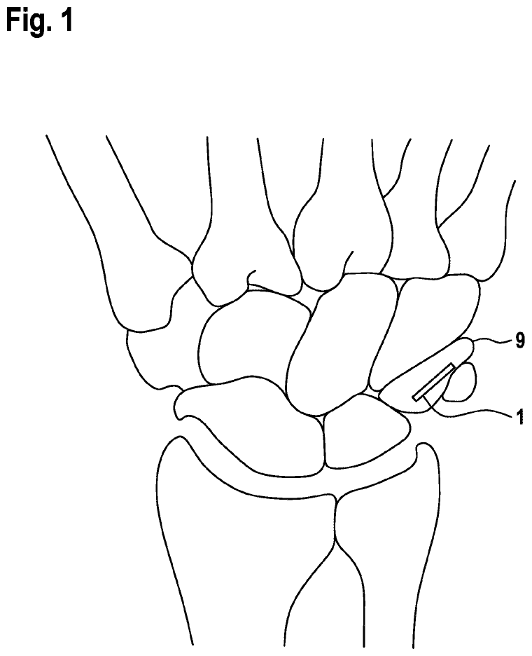

Biocorrodible solid body 1 comprises different areas 5, 6 that differ from one another in terms of their corrosion behavior. The different corrosion behavior of different areas 5, 6 is largely determined by the different nature of coating 3 in different areas 5, 6. Here, slower biocorrosion occurs in a coating section 7, but faster biocorrosion occurs in coating section 8. The different rates of corrosion attack in different areas 5, 6 are illustrated in FIGS. 1 to 3. These figures show a metallic solid body 1 inserted into a bone 9, which solid body 1 is a magnesium-based implant.

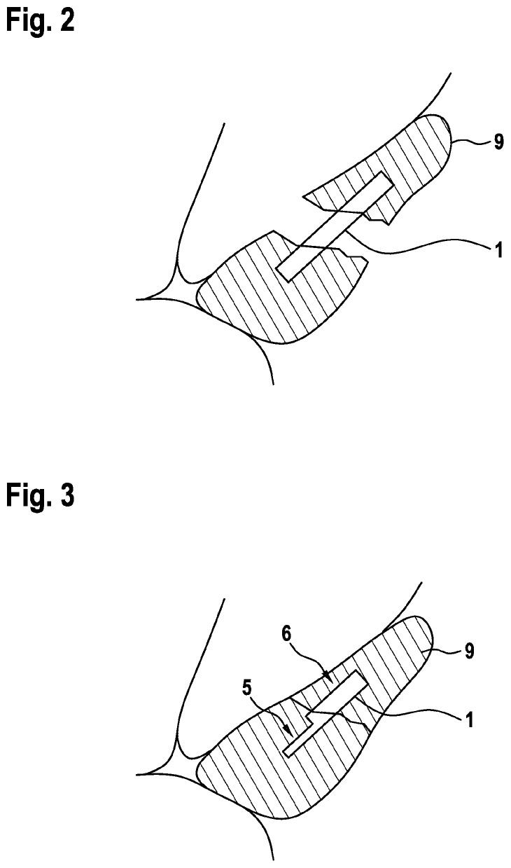

FIG. 2 illustrates a state during implantation of solid body 1, FIG. 3 illustrates the state after partial degradation of solid body 1. As can be seen from a comparison of FIGS. 2 and 3, the material thickness of solid body 1 in the state visualized in FIG. 3 has already decreased significantly a few weeks after surgery (FIG. 2). In area 6, however, hardly any biocorrosion had occurred up to this point. Two desired effects are associated with this splitting of the corrosion rates: On the one hand, the substance resulting from biocorrosion, especially hydrogen, is released over a longer period of time at a rate that is never too high. On the other hand, the anchoring of area 6 in bone 9 is maintained over a longer period of time. After complete healing, entire implant 1 is replaced by bone material.

FIG. 4 illustrates the nature of the various sections 7, 8 of coating 3 of implant 1 used in the example according to FIGS. 1 to 3. Porosity can be seen in both section 7 and section 8 of coating 3, with pores being designated 10, 11 and present in different sizes and distribution in different sections 7, 8. The different nature of coating 3 in different sections 7, 8 is generally not visible to the naked eye. However, the different distribution and average size of pores 10, 11 ensures a significantly different biocorrosion behavior of different sections 7, 8 in the given biological environment. This is due in particular to the different hydrophilic or hydrophobic properties of coating sections 7, 8. In coating section 7, which is characterized by low corrosion attack, coating 3 has hydrophobic properties, which ensure long-term protection of base material 2, with hardly any oxygen permeability of coating 3 in section 7. In contrast, in section 8 of coating 3, which has a higher porosity, hydrophilic properties are set, which are accompanied by more rapid biocorrosion.

FIG. 5 shows an alternative possibility of setting different biocorrosion properties in different areas 5, 6 of solid body 1. This variant is also suitable for the application according to FIGS. 1 to 3. In this case, various sections 7, 8 of coating 3 have a uniform composition and only differ from one another in terms of their thickness D₃ₘᵢₙ, D₃ₘₐₓ. In contrast to the example outlined in FIG. 4, base material 2 and thus entire solid body 1 in the case of FIG. 5 have a non-uniform material thickness. There is a negative correlation between the thickness of coating 3 and the thickness of base material 2. This means that coating 3 is thinnest where base material 2 is thickest. The corrosion of base material 2 thus begins in its thickest area 5. In comparatively thin area 6 of implant 1, however, biocorrosion occurs with a greater delay. Ultimately, this ensures that various areas 5, 6 are completely degraded at approximately the same time. Deviating from the simplified representation according to FIG. 5, more than two different sections 7, 8 of coating 3 can also be formed or there can be continuous transitions between different coating sections 7, 8. This also applies in an analogous manner to the material properties of various coating sections 7, 8 of implant 1 according to FIG. 4.

### LIST OF REFERENCE NUMERALS

1 solid body
2 base material
3 coating
4 biocidal substance
5 area of the solid body
6 area of the solid body
7 surface section with slower biocorrosion
8 surface section with faster biocorrosion
9 bone
10 pore
11 pore
D₁, D₃, D₁ₘᵢₙ, D₁ₘₐₓ, D₃ₘᵢₙ, D₃ₘₐₓ thickness

1. A biocorrodible solid body, with a base material and a coating, the coating comprising silicon and being applied to the base material so that different corrosion rates are established in different areas of the solid body, the different areas differing from one another with regard to at least one of properties porosity, thickness, hydrophilic/hydrophobic properties and composition.

2. The solid body according to claim 1, wherein the solid body is an implant.

3. The solid body according to claim 1, comprising a biocidal substance located between the base material and the coating.

4. The solid body according to claim 1, wherein the base material and the coating have a porous structure.

5. The solid body according to claim 1, wherein biodegradability of the coating is positively correlated with a thickness of the base material.

6. The solid body according to claim 1, wherein biodegradability of the coating is negatively correlated with thickness of the base material.

7. The solid body according to claim 1, wherein the base material contains magnesium as a main component.

8. The solid body according to claim 1, wherein the base material comprises a polymer.

9. The solid body according to claim 1, wherein the base material has a uniform structure in contrast to the coating.

10. The solid body according to claim 1, comprising a fiber or fabric.

11. The solid body according to claim 1, comprising a biological material.

12. A method of using the solid body according to claim 2, comprising implanting the implant in hand surgery or foot surgery.

13. A method for coating a solid body, comprising an implant, wherein a biocidal substance and a silicon-containing coating are applied to a base material in a non-uniform manner on various surface sections so that different areas differ from each other with regard to at least one of porosity, thickness, hydrophilic/hydrophobic properties and composition, wherein biodegradability in individual areas of the implant, is set differently.

14. The method according to claim 13, wherein the biocidal substance is first applied in a uniform manner to an entire surface of the base material and in a further method step the silicon-containing coating is applied to different surface sections in a non-uniform manner.

15. The method according to claim 13, wherein both the biocidal substance and the silicon-containing coating are applied to different surface sections of the base material in a non-uniform manner.

16. The method according to claim 13, wherein the biocidal substance and the further coating are applied so that the biocidal substance is only released during corrosion of the coating.

17. The method according to claim 13, wherein a structure of the coating is configured to be impermeable to oxygen at least at a beginning of use.

18. The solid body according to claim 1, wherein the base material comprises a polylactide, polysaccharide, polyamide, polyester, PLGA or PCL.

19. The method according to claim 13, wherein the implant comprises a magnesium implant.
